(19) United States
(12) Patent Application Publication
(10) Pub. No.: US 2013/0041263 A1
(43) Pub. Date: Feb. 14, 2013

(54) METHOD OF DETECTING A SUICIDE TERRORIST

(71) Applicant: Ming-Ta WU, Taipei City (TW)

(72) Inventor: Ming-Ta WU, Taipei City (TW)

(21) Appl. No.: 13/204,940

(22) Filed: Aug. 8, 2011

(51) Int. Cl. A61B 6/00; A61B 5/026

(52) U.S. Cl. 600/473; 600/504

(57) ABSTRACT

A method of detecting a suicide terrorist, comprises: (A) selecting a test subject; (B) installing a testing device that can detect real-time variations in cerebral blood flows at a first tested area and second tested area of the subject, wherein the first tested area corresponds to a suicide intent area of a cortex, and the second tested area corresponds to a negative response area of the cortex; (C) using the testing device to detect cortex activity information related to the cerebral blood flow variations of the first tested area within a predetermined time; (D) comparing the cortex activity information with a control group associated with normal individuals to obtain a result; (E) according to the result of step (D), determining whether the subject has suicide intent; (F)asking whether the subject is a terrorist; (G) using the testing device to detect a cortex activity information related to the cerebral blood flow variations of the second tested area within a predetermined time to obtain a result; and (H) according to the result of step (G), determining whether the subject is a terrorist. This method can be advantageously employed at various security check sites, such as airports, train stations, or any other sites susceptible of terrorist attacks.

### BACKGROUND OF THE INVENTION

1. Field of the Invention

The present invention relates to a method of detecting suicide intent, and more particularly to a method of detecting a suicide terrorist.

2. The Prior Arts

In the “September 11” terrorist attack against the United States, four airplanes were hijacked and redirected to collide against the two towers of the World Trade Center and the building of the Pentagon, which caused thousands of civilian victims. This kamikaze attack shocked many countries around the world, and revealed unconventional war methods that use suicide attacks against symbolic sites to cause mass killing of civilian population, with the goal of creating fear and destabilizing the enemy country.

In the war against terrorists that followed this event, a decisive turn occurred on May 1, 2011 when the most well-known terrorist leader, Ousama bin Laden, was killed by a U.S. commando in Pakistan. However, there is still fear that terrorist groups may conduct revenge attacks. Therefore, there is a need for effective and efficient security check methods that can be conducted in public sites (such as airports) or locations of large civilian concentration to prevent suicide terrorist attacks.

### SUMMARY OF THE INVENTION

A conventional security check mainly consists of examining personal background information, or using scanning apparatuses. This traditional procedure cannot directly detect the real intention of a terrorist to perform a suicide attack. Moreover, terrorist groups become increasingly sophisticated to find flaws and deceive the security check procedure.

One aspect of the present invention is to provide a method that can efficiently detect a suicide terrorist. According to the present invention, the method comprises: (A) selecting a test subject; (B) installing a testing device that can detect real-time variations in cerebral blood flows at a first tested area and second tested area of the subject, wherein the first tested area corresponds to a suicide intent area of a cortex, and the second tested area corresponds to a negative response area of the cortex; (C) using the testing device to detect cortex activity information related to the cerebral blood flow variations of the first tested area within a predetermined time; (D) comparing the cortex activity information with a control group associated with normal individuals to obtain a result; (E) according to the result of step (D), determining whether the subject has suicide intent; (F) asking whether the subject is a terrorist; (G) using the testing device to detect cortex activity information related to the cerebral blood flow variations of the second tested area within a predetermined time to obtain a result; and (H) according to the result of step (G), determining whether the subject is a terrorist.

With the method of the present invention, the activity of specific brain areas in the subject's head can be detected within seconds to determine the attack intent of the terrorist. Furthermore, this method can be combined with lie detection techniques to detect psychologically weaker individuals having suicide intent and trained terrorists. The method according to the present invention can be advantageously employed at various security check sites, such as airports, train stations, or any other sites susceptible of terrorist attacks.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a flowchart of a method of detecting a suicide terrorist according to the present invention;

FIG. 2 is a schematic view showing a first tested area in a cortex;

FIG. 3 is a schematic view showing a second tested area in a cortex; and

FIGS. 4A and 4B are comparison diagrams showing significant difference results in the first tested area when compared to a control group of normal individuals.

### DETAILED DESCRIPTION OF THE PREFERRED EMBODIMENTS

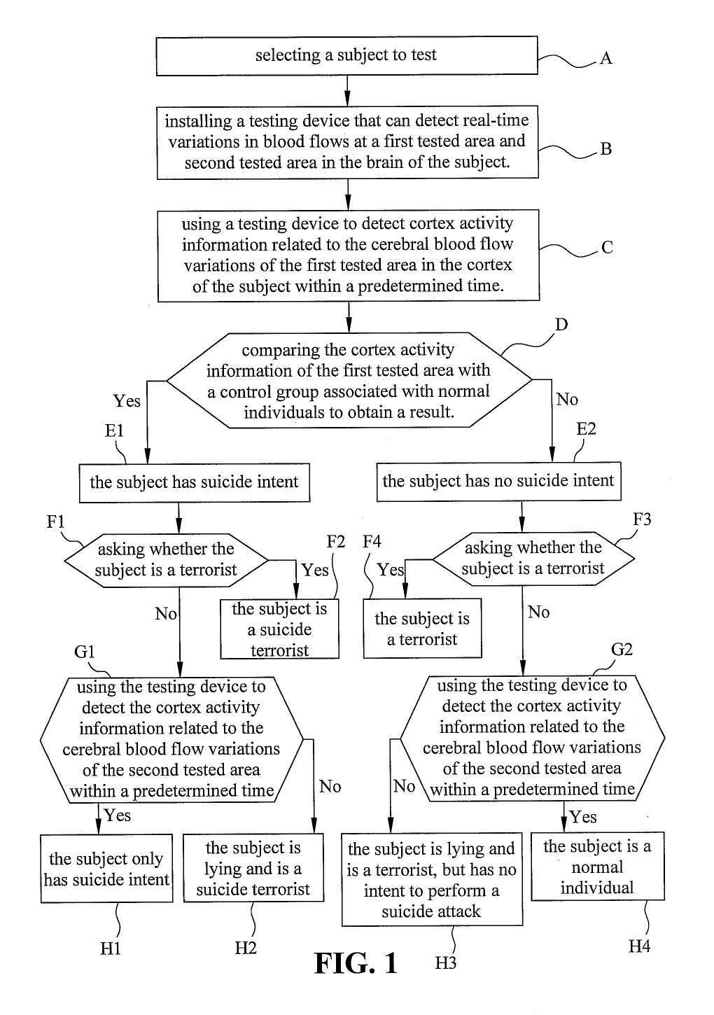

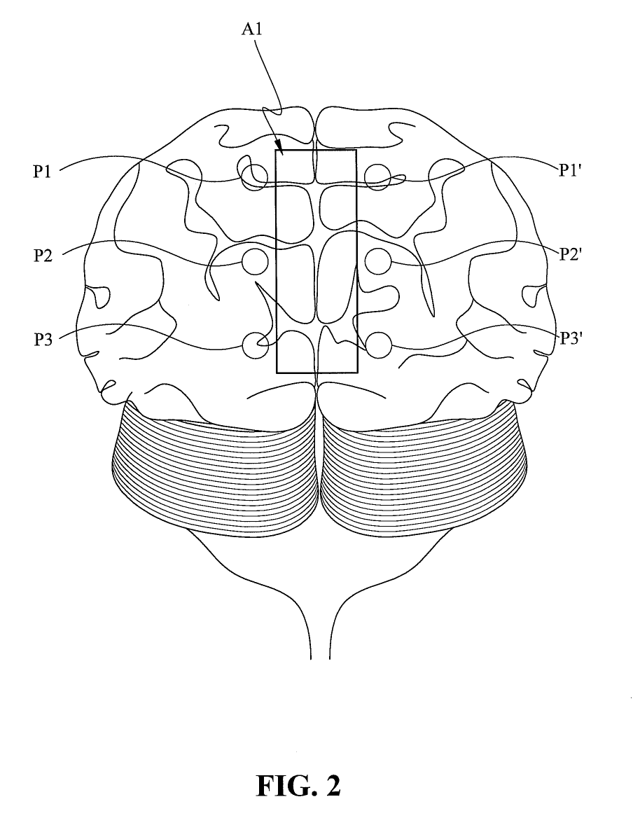

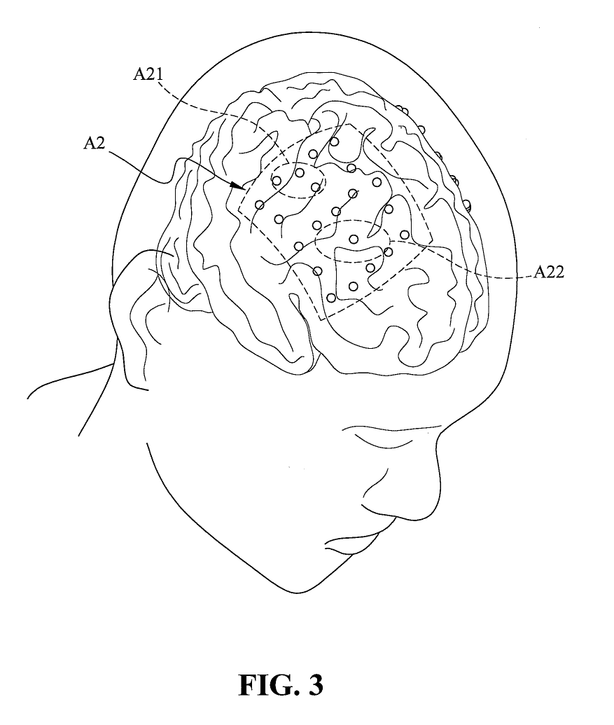

FIG. 1 is a flowchart of a method of detecting a suicide terrorist according to the present invention, FIG. 2 is a schematic view showing a tested area of a cortex, and FIG. 3 is a schematic view showing a negative response area in a cortex. As shown, the method comprises the following steps.

Step (A): selecting a test subject. The subject may be specifically selected or randomly selected. The test subject may be chosen according to personal profile data or an apparent physiologic state, and by evaluating the answer provided by an individual to an inquiry about the purpose of his/her trip. For example, the subject may be selected among passengers at the immigration counter of an airport according to intelligence information, background information and/or travel countries showing that he/she may be a terrorist, or according to a physiologic state, such as nervous, sweating and/or shaking reactions, or any other suspicious reactions. The selected subject can then undergo the following testing steps.

Step (B): installing a testing device that can detect real-time variations in cerebral blood flows at a first tested area and second tested area of the subject. The first tested area corresponds to a suicide intent area of the cortex, and the second tested area corresponds to a negative response area of the cortex. The aforementioned testing device can be a near infrared reflectance spectroscopy (NIRS) device, or other devices using different testing principles but have the same functions. Moreover, the testing device can be a stationary or portable testing device. If testing convenience is required, it may be preferable to use a portable NIRS device. As shown in FIG. 2, the first tested area A1 can be a rectangular area having a length of about 10 cm and a width of about 4 cm. The suicide intent area is a specific region of the cortex that was actually detected by the inventor of the present application and never disclosed to the public. Therefore, the name of the inventor is used to designate this region as “Wu-type suicide area.”

Step (C): using the testing device to detect cortex activity information related to the cerebral blood flow variations of the first tested area within a predetermined time. The NIRS device can sense light reflectance from the subject's head to detect real-time variations of the blood flow occurring in the cortex without being affected by electromagnetic noise, which can provide high resolution measures. As shown in FIG. 2, the testing device may be installed by exemplary placing three pairs of NIRS contact points at the periphery of the first tested area Al, which can include signal emission points P1, P2, P3, and signal detection points P1′, P2′, P3′, the left and right positions being interchangeable. In this step, the predetermined time may be preferably between 1 and 10 seconds. However, the testing time is not limited to the above duration, and may be adjusted according to the actual conditions.

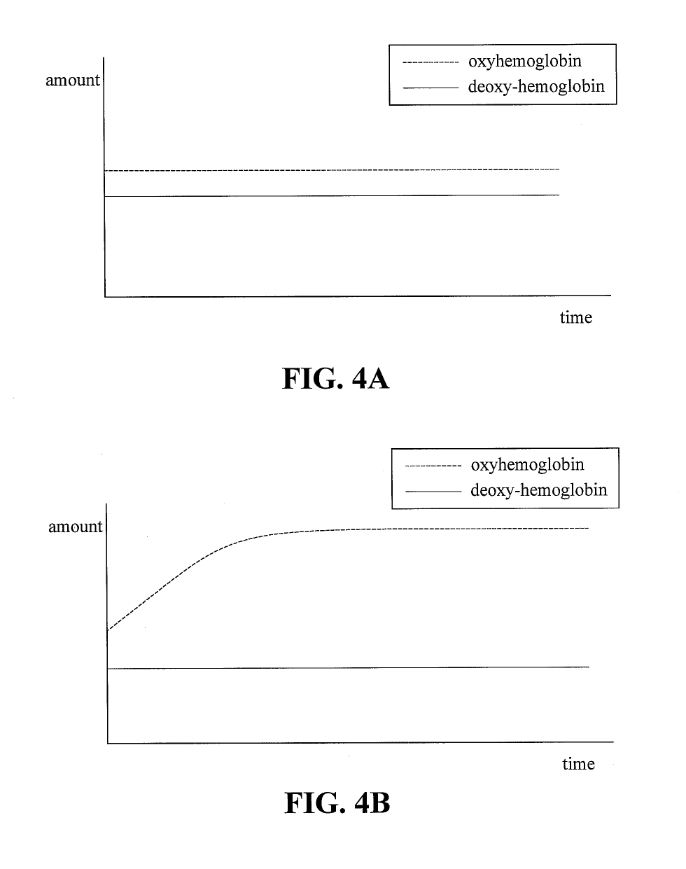

Step (D): comparing the cortex activity information with a control group associated with normal individuals to obtain a result. The above variations in the cerebral blood flow can be compared with the variation in the cerebral blood flow of a normal individual. FIGS. 4A and 4B schematically show significant differences that may be detected in this step.

Steps (E1) and (E2): according to the result of step (D), determining whether the subject has suicide intent. In other words, according to the result of the previous comparison step, it is determined whether the first tested area is in an active condition. Step (E1): if there is a significant difference, it means that the tested subject has suicide intent. Step (E2): if there is no significant difference, it means that the tested subject does not have any suicide intent. Subsequently, lie detection techniques can be used to determine whether the tested subject with/without suicide intent is a terrorist.

Steps (F1) and (F3): asking whether the subject is a terrorist. If the response after step (F1) is “yes”, step (F2) is performed, which means that the tested subject is a suicide terrorist. If the response after step (F3) is “yes”, step (F4) is performed, which means that the tested subject is a terrorist but not a suicide terrorist.

If the response after step (F1) is “no”, step (G1) is performed. In step (G1), the testing device is used to detect the cortex activity information related to the cerebral blood flow variations of the second tested area in the subject's cortex within a predetermined time (1 to 10 seconds). As shown in FIG. 3, the second tested area A2 is a negative response area of the cortex, which includes two negative response areas A21 and A22 of the cortex. By detecting the activity of the second tested area, it can be determined whether the negative response of the tested subject is a lie. This step mainly combines near infrared spectroscopy techniques and lie detection methods for the negative response area in the cortex. The principle of this method is to detect the activity of the negative response area in the cortex when the subject answers a question with a negative response, and accordingly determine whether the negative response is a true reply. If the negative response area is significantly active, it means that the tested subject is telling the truth and only has suicide intent as shown in step (H1). If the negative response area does not exhibit significant activity, it means that the tested subject is lying and is a suicide terrorist as shown in step (H2).

On the other hand, if the response after step (F3) is “no”, step (G2) is performed. In step (G2), the testing device is used to detect the cortex activity information related to the cerebral blood flow variations of the second tested area in the subject's cortex within a predetermined time (1 to 10 seconds). It can be thereby determined whether the negative response of the subject is a lie. If the negative response area does not exhibit significant activity, it means that the subject is lying and is a terrorist as shown in step (H3), but has no intent to use suicide attack. If the negative response area exhibit significant activity, it means that the subject is telling the truth and is a normal individual as shown in step (H4). With the detection method of this embodiment, the tested subject can be distinguished between 4 categories including: normal individual, a normal individual having suicide intent, terrorist, and suicide terrorist. Therefore, security check can be facilitated. As described above, the method of detecting suicide terrorist provided by the present invention has industrial value.

The foregoing description is intended to only provide illustrative ways of implementing the present invention, and should not be construed as limitations to the scope of the present invention. While the foregoing is directed to embodiments of the present invention, other and further embodiments of the invention may thus be devised without departing from the basic scope thereof, and the scope thereof is determined by the claims that follow.

What is claimed is:

1. A method of detecting a suicide terrorist, comprising:
(A) selecting a test subject;
(B) installing a testing device that can detect real-time variations in cerebral blood flows at a first tested area and second tested area of the subject, wherein the first tested area corresponds to a suicide intent area of a cortex, and the second tested area corresponds to a negative response area of the cortex;
(C) using the testing device to detect cortex activity information related to the cerebral blood flow variations of the first tested area within a predetermined time;
(D) comparing the cortex activity information with a control group associated with normal individuals to obtain a result;
(E) according to the result of step (D), determining whether the subject has suicide intent;
(F) asking whether the subject is a terrorist;
(G) using the testing device to detect cortex activity information related to the cerebral blood flow variations of the second tested area within a predetermined time to obtain a result; and
(H) according to the result of step (G), determining whether the subject is a terrorist.

2. The method of claim 1, wherein the subject is specifically or randomly selected in step (A).

3. The method of claim 2, wherein the subject is selected according to personal background information or an apparent physiologic state of the subject.

4. The method of claim 1, wherein step (A) further comprises asking the subject about a travel purpose.

5. The method of claim 1, wherein the testing device used in step (B) is a near-infrared spectroscopy device for cerebral blood flow detection.

6. The method of claim 1, wherein the first tested area in step (B) has a rectangular shape with a length of 10 cm and a width of 4 cm.

7. The method of claim 1, wherein the predetermined time in steps (C) and (G) is between 1 second and 10 seconds.

8. The method of claim 1, wherein the subject has suicide intent if the result of step (E) shows a significant difference.

9. The method of claim 8, wherein the subject only has suicide intent if the result of step (H) exhibits significant activity.

10. The method of claim 8, wherein the subject is lying and is a suicide terrorist if the result of step (H) does not exhibit significant activity.

11. The method of claim 1, wherein the subject does not have any suicide intent if the result of step (E) shows no significant difference.

12. The method of claim 11, wherein step (H) represents that the subject is a normal individual if the result of step (G) exhibits a significant activity.

13. The method of claim 11, wherein step (H) represents that the subject is lying and is a terrorist without suicide attack intent if the result of step (G) exhibits no significant activity.
